(19) United States
(12) Patent Application Publication
(10) Pub. No.: US 2012/0077269 A1
(43) Pub. Date: Mar. 29, 2012

(54) USE OF A PROTEIN IN STEM CELL AND CANCER APPLICATIONS

(75) Inventors: Olle Nilsson, Goteborg (SE); Karin Majnesjö, Olofstorp (SE); Johan Hyllner, Goteborg (SE); Raimund Strehl, Goteborg (SE); Eva Kilmare, Goteborg (SE); Fredrik Wessberg, Goteborg (SE)

(73) Assignee: CELLARTIS AB, Göteborg (SE)

(21) Appl. No.: 13/142,909

(22) PCT Filed: Dec. 30, 2009
(86) PCT No.: PCT/SE09/00546

§ 371 Date: Sep. 23, 2011

(30) Foreign Application Priority Data
Dec. 30, 2008 (SE) 0802680-9
Apr. 17, 2009 (SE) 0900523-2

(51) Int. Cl. C12N 5/071; C07K 14/705

(52) U.S. Cl. 435/366; 530/395; 530/324

(57) ABSTRACT

The present invention discloses a novel antibody HES5:3:3 directed towards a specific antigen present on human pluripotent stem (hPS) cells and cancer tissue. The antibody can be used as a tool for human pluripotent stem (hPS) cell applications, such as the separation, surface adhesion and enhanced survival of said hPS cells. Furthermore, the present invention refers to the use of the antigen detection for cancer applications.

### TECHNICAL FIELD

The present invention relates to a novel antibody HES5:3:3 directed towards a splice variant of the basigin (CD147) protein and uses thereof for human pluripotent stem (hPS) cell applications, such as separation, surface adhesion and enhanced survival of said hPS cells. Furthermore, the present invention refers to the use of the detection the splice variant of basigin for cancer applications.

### BACKGROUND OF THE INVENTION

Human pluripotent stem (hPS) cells are derived from the inner cell mass (ICM) of preimplantation blastocysts and have the unique ability to proliferate indefinitely in-vitro in an undifferentiated state. Due to their pluripotent nature, hPS cells offer the possibility to derive well-defined, functionally differentiated cells for in-vitro applications in drug discovery and toxicology in the short term as well as regenerative medicine in the longer term since hPS cells are excellent candidates to serve as a valuable source of cells in transplantation medicine. Fulfilling this potential requires extensive development of methods and reagents for studying hPS cell self-renewal and differentiation. One fundamental requirement for using hPS cells in the above mentioned applications is that the population used is homogenous and free from unwanted cells.

Traditionally hPS cell lines were cultivated on mitotically inactivated mouse embryonic fibroblasts (MEFs). hPS cells derived in this manner became contaminated by animal cells and were therefore unsuitable for use in clinical applications. Due to this disadvantage, the improvement of cultivating the hPSs on human feeder systems which efficiently support undifferentiated growth of hPS cells was developed. Unfortunately, this improved cultivation method resulted in hPS cell populations contaminated with feeder cells. Other factors resulting in inhomogeneous hPS cell populations are spontaneous differentiations [Sathananthan A H, Trounson A. Human embryonic stem cells and their spontaneous differentiation. Ital J. Anat Embryol. 2005; 110 (2 Suppl 1):151-7. Review] and potential alterations during extended culture [Park Y B, Kim Y Y, Oh S K et al. Alterations of proliferative and differentiation potentials of human embryonic stem cells during long-term culture, Exp Mol. Med. 2008 Feb. 29; 40(1):98-108]. Therefore, there is need for an efficient and highly specific tool for the exclusive separation of contaminating feeder and spontaneously differentiated cells from live undifferentiated hPS cells.

The most selective markers of undifferentiated stem cell state used to date are transcription factors Oct-4 [Schöler H R, Hatzopoulos A K, Balling R et al, A family of octamer-specific proteins present during mouse embryogenesis: evidence for germline-specific expression of an Oct factor EMBO J. 1989 September; 8(9):2543-50] and Nanog [Chambers I, Colby D, Robertson M et al. Functional expression cloning of Nanog, a pluripotency sustaining factor in embryonic stem cells. [Cell. 2003 May 30; 113(5):643-55] which are present in the nucleus. Due to their localization in the nucleus, antibodies against these epitopes can't be used for magnetic separation or fluorescence activated sorting. Cell surface specific antigens commonly used to confirm the undifferentiated state of hPS cells either by immunocytochemistry or by flow cytometry analysis are stage specific embryonic antigen 1 (SSEA-1) [Solter D and Knowles B B. Monoclonal antibody defining a stage-specific mouse embryonic antigen (SSEA-1). Proceedings of the National Academy of Sciences (USA) 75: 5565-5569 (1978)], SSEA-3 [Shevinsky L H, Knowles B B, Damjanov I et al. Monoclonal antibody to murine embryos defines a stage-specific embryonic antigen expressed on mouse embryos and human teratocarcinoma cells. Cell. 1982 October; 30(3):697-705] and SSEA-4 [Kannagi R, Cochran N A, Ishigami F et al. Stage-specific embryonic antigens (SSEA-3 and -4) are epitopes of a unique globo-series ganglioside isolated from human teratocarcinoma cells. EMBO J. 1983; 2(12):2355-61] tumour receptor antigens 1-60 (TRA1-60) and TRA1-81 [Andrews P W, Banting G, Damjanov I et al. Three monoclonal antibodies defining distinct differentiation antigens associated with different high molecular weight polypeptides on the surface of human embryonal carcinoma cells. Hybridoma. 1984 Winter; 3(4):347-61]. These markers were originally established for mouse embryonic stem (mES) or for embryocarcinoma (EC) cells and therefore not exclusively specific for undifferentiated human ES cells.

Only a few monoclonal antibodies (mAbs) have been developed specifically for the characterization, manipulation and purification of hPS cells [Choo A B, Tan H L, Ang S N et al. Selection against undifferentiated human embryonic stem cells by a cytoxic antibody recognizing podocalyxin-like protein-1. Stem Cells Express, first published online Mar. 20, 2008; doi: 10.1634/stemcells.2007-0576, Son Y S, Park J H, Kang Y K et al. Heat shock 70-kDa protein 8 isoform 1 is expressed on the surface of human embryonic stem cells and downregulated upon differentiation. Stem Cells. 2005, Oct; 23:1502-1513], and therefore one object of this invention is to expand the repertoire of antibodies against unknown antigens found exclusively on the surface of undifferentiated hPS cells.

In this regard, mice were immunized with a mixture of undifferentiated hPS cell antigens e.g. whole hPS cells where the cell membrane with surface antigens were kept as intact as possible, and conventional hybridoma technology was used to establish hybridoma cell lines. The desired candidates were subsequently selected in a tiered screening approach. The chosen candidate hybridomas were subcloned, the produced antibodies purified and characterized. This approach led to the successful generation of the highly specific antibody, HES5:3:3 against a surface antigen on the undifferentiated hPS cells. Further experimental data demonstrate that MAb HES5:3:3 targets an epitope within the extra 115 amino acids of Basigin isoform 1, which is the longer splice variant of Basigin isoform 2 (also known as CD147 or Emmpirin). Notably, the antibody HES5:3:3 is shown to be more responsive to early differentiation than commonly used pluripotency marker Oct-4 and display less cross-reactivity to feeder cells than the SSEA and TRA markers. In addition, the generated antibody was successfully used for staining of live cells (e.g. non-fixated) and magnetic separation of undifferentiated hPS cells from contaminating feeder or spontaneously differentiated cells. Therefore, the present invention provides a novel monoclonal antibody that can be used as a valuable tool for the characterization of pluripotent cell populations due to its high specificity, negative response to early differentiation and suitability for effective magnetic separation of hPS cells.

Markers specific for undifferentiated hPS cells are also potential oncofetal antigens. Oncofetal antigens are proteins, which are expressed at different stages during development of the fetus, and are re-expressed in certain tumours in adults but are not present, or are expressed at significantly lower levels in the corresponding normal adult tissue. The oncofetal antigens have the potential to be used as tumour markers for diagnosis of malignancy, treatment monitoring, follow-up after therapy and ultimately as targets for specific therapy of malignancy. One such marker is for example the alpha-fetoprotein, which is highly associated with hepatocellular carcinoma. The present invention demonstrates that the same MAb HES5:3:3 used in the characterization of pluripotent cell populations can also be used to identify many carcinomas of different organs. WO2008007648 discloses an antibody against CD147 (Basigin isoform 2) that has been selected from a phage display antibody library. The antibody was selected for binding to HepG2 cells and immuno-precipitation followed by mass-spectrometry analysis identified the antigen as CD147. RNAi studies confirmed the identity of the target antigen. However, the binding epitope on CD147 was not determined and it is not shown if the antibody targets a region present in all splice variants of Basigin or if it is specific for a particular variant. This is in contrast to MAb HES5:3:3 which has been shown to target an epitope within the unique segment of the long splice variant (Basigin isoform 1).

### SUMMARY OF THE INVENTION

A first aspect of the invention relates to the use of a polypeptide represented by the following amino acid sequence MAAALFVLLG FALLGTHGAS GAAGFVQAPL SQQRWVGGSV ELHCEAVGSP VPEIQWWFEG QGPNDTCSQL WDGARLDRVH IHATYHQHAA STISIDTLVE EDTGTYECRA SNDPDRNHLT RAPRVKWVRA QAVVLVLEPG TVFTTVEDLG SKILLTCSLN DSATEVTGHR WLKGGVVLKE DALPGQKTEF KVDSDDQWGE YSCVFLPEPM GTANIQLHGP PRVKAVKSSE HINEGETAML VCKSESVPPV TDWAWYKITD SEDKALMNGS ESRFFVSSSQ GRSELHIENL NMEADPGQYR CNGTSSKGSD QAIITLRVRS HLAALWPFLG IVAEVLVLVT IIFIYEKRRK PEDVLDDDDA GSAPLKSSGQ HQNDKGKNVR QRNSS (SEQ ID NO 1), or a polypeptide consisting of a partial sequence of an amino acid sequence represented by SEQ ID NO 1, as a tool for human stem cell applications involving stem cells of the group comprising pluripotent stem cells, multipotent stem cells, oligopotent stem cells and/or unipotent cells.

In a preferred embodiment of the invention the invention relates to the use of one or more of the following partial sequences

(SEQ ID NO 9)
AAGFVQAPLSQQRWVGGSVELHCEAVGSPVPEIQWWFEGQ,
(SEQ ID NO 10)
PEIQWWFEGQGPNDTCSQLWDGARLDRVHIHATYHQHAAS,
(SEQ ID NO 11)
HATYHQHAAS QHAASTISIDTLVEEDTGTYECRASNDPDRNHLTR,
or
(SEQ ID NO 12)
NDPDRNHLTRAPRVKWVRAQAVVLVLEPGTVFTTVED.

In a particularly preferred embodiment of the invention relates to the use of the partial sequence HATYHQHAAS QHAASTISIDTLVEEDTGTYECRASNDPDRNHLTR (SEQ ID NO 11).

In one embodiment the preferred polypeptide is used as a marker to selectively mark, sort or separate human stem cell of the group comprising pluripotent stem cells, multipotent stem cells, oligopotent stem cells, unipotent cells, human germ cells, such as sperm cells, human progenitor cells of the group comprising early differentiated cell types derived from human stem cells, early differentiated cell types derived from human pluripotent stem cells (hPSC) and/or undifferentiated human pluripotent stem cells (hPSC).

In another embodiment the preferred polypeptide is used as a media supplement or culture vessel coating for the enhanced survival and adhesion of human pluripotent stem cells (hPSC) during culturing.

In another embodiment the preferred polypeptide is used for the down regulation of the differentiation process in human pluripotent stem cells (hPSC) in non-supportive culture conditions.

In another preferred embodiment of the invention the polypeptide is used as a tumour marker when said polypeptide is present in cancer tissue and the cancer tissue is selected from one or more tissues of the group comprising carcinomas of the kidney, stomach, colon/rectum, larynx, lung, ovary, bladder and/or thyroid.

A second aspect of the invention relates to an isolated polypeptide characterized in that it is represented by the following amino acid sequence: MAAALFVLLG FALLGTHGAS GAAGFVQAPL SQQRWVGGSV ELHCEAVGSP VPEIQWWFEG QGPNDTCSQL WDGARLDRVH IHATYHQHAA STISIDTLVE EDTGTYECRA SNDPDRNHLT. RAPRVKWVRA QAVVLVLEPG TVFTTVEDLG SKILLTCSLN DSATEVTGHR WLKGGVVLKE DALPGQKTEF KVDSDDQWGE YSCVFLPEPM GTANIQLHGP PRVKAVKSSE HINEGETAML VCKSESVPPV TDWAWYKITD SEDKALMNGS ESRFFVSSSQ GRSELHIENL NMEADPGQYR CNGTSSKGSD QAIITLRVRS HLAALWPFLG IVAEVLVLVT IIFIYEKRRK PEDVLDDDDA GSAPLKSSGQ HQNDKGKNVR QRNSS (SEQ ID NO 1), or a polypeptide consisting of a partial sequence of the amino acid sequence represented by SEQ ID NO 1.

In a preferred embodiment of the invention the partial sequence is one or more of the following sequences AAGFVQAPLSQQRWVGGSVELHCEAVGSPVPEIQWWFEGQ (SEQ ID NO 9), PEIQWWFEGQGPNDTCSQLWDGARLDRVHIHATYHQHAAS (SEQ ID NO 10), HATYHQHAAS QHAASTISIDTLVEEDTGTYECRASNDPDRNHLTR (SEQ ID NO 11), or NDPDRNHLTRAPRVKWVRAQAVVLVLEPGTVFTTVED (SEQ ID NO 12).

In a particularly preferred embodiment of the invention the partial sequence is HATYHQHAASQHAASTISIDTLVEEDTGTYECRASNDPDRNHLTR (SEQ ID NO 11).

In a preferred embodiment of the invention the polypeptide selectively binds to the monoclonal antibody produced by the hybridoma HES5:3:3 cell line having the ECACC accession number 08111901.

In a preferred embodiment of the invention the polypeptide is expressed on the cell surface.

In one embodiment of the invention the polypeptide is expressed on the cell surface of human stem cells of the group comprising pluripotent stem cells, multipotent stem cells, oligopotent stem cells and/or unipotent cells, human germ cells, such as sperm cells, human progenitor cells of the group comprising early differentiated cell types derived from human stem cells, early differentiated cell types derived from human pluripotent stem cells (hPSC) and/or undifferentiated human pluripotent stem cells (hPSC).

In a preferred embodiment of the invention the polypeptide is expressed on the cell surface of cancer tissue cells and in particular when the cancer tissue is selected from one or more tissues of the group comprising carcinomas of the kidney, stomach, colon/rectum, larynx, lung, ovary, bladder and/or thyroid.

A third aspect of the invention relates to an antibody or an antigen binding fragment thereof, that specifically binds a polypeptide represented by the amino acid sequence MAAALFVLLG FALLGTHGAS GAAGFVQAPL SQQRWVGGSV ELHCEAVGSP VPEIQWWFEG QGPNDTCSQL WDGARLDRVH IHATYHQHAA STISIDTLVE EDTGTYECRA SNDPDRNHLT RAPRVKWVRA QAVVLVLEPG TVFTTVEDLG SKILLTCSLN DSATEVTGHR WLKGGVVLKE DALPGQKTEF KVDSDDQWGE YSCVFLPEPM GTANIQLHGP PRVKAVKSSE HINEGETAML VCKSESVPPV TDWAWYKITD SEDKALMNGS ESRFFVSSSQ GRSELHIENL NMEADPGQYR CNGTSSKGSD QAIITLRVRS HLAALWPFLG IVAEVLVLVT IIFIYEKRRK PEDVLDDDDA GSAPLKSSGQ HQNDKGKNVR QRNSS (SEQ ID NO 1), or a polypeptide consisting of a partial sequence of an amino acid sequence represented by SEQ ID NO 1, said antibody being produced by the hybridoma HES5 cell line having the ECACC accession number 08111901.

In a preferred embodiment of the invention the antibody or an antigen binding fragment thereof binds to one or more of the following sequences AGFVQAPLSQQRWVGGSVELH CEAVGSPVPEIQWWFEGQ (SEQ ID NO 9), PEIQWWFEGQGPNDTCSQLWDGARLD RVHIHATYHQHAAS (SEQ ID NO 10), HATYHQHAASQHAASTISIDTLVEEDTGTYECRA SNDPDRNHLTR (SEQ ID NO 11), or NDPDRNHLTRAPRVKWVRAQAVVLVLEPG TVFTTVED (SEQ ID NO 12).

In a preferred embodiment of the invention the antibody or an antigen binding fragment thereof binds to the partial sequence HATYHQHAASQHAASTISIDTLVEEDTGTYE CRASND PDRNHLTR (SEQ ID NO 12).

In a preferred embodiment of the invention the antibody or an antigen binding fragment thereof is a monoclonal antibody.

In a preferred embodiment of the invention the antibody or an antigen binding fragment thereof is an IgG antibody.

In a preferred embodiment of the invention the antibody or an antigen binding fragment thereof specifically binds human stem cells of the group comprising pluripotent stem cells, multipotent stem cells, oligopotent stem cells, unipotent cells, human germ cells, such as sperm cells, human progenitor cells of the group comprising early differentiated cell types derived from human stem cells, early differentiated cell types derived from human pluripotent stem cells (hPSC), undifferentiated human pluripotent stem cells (hPSC).

In a preferred embodiment of the invention the antibody or an antigen binding fragment thereof is coupled to a magnetic bead.

In a preferred embodiment of the invention the antibody or an antigen binding fragment thereof is marked with a fluorescent probe.

In a preferred embodiment of the invention the antibody or an antigen binding fragment thereof selectively binds to cancer tissue and in particular one or more cancer tissues selected from the group comprising carcinomas of the kidney, stomach, colon/rectum, larynx, lung, ovary, bladder and/or thyroid.

A fourth aspect of the invention relates to the use of the antibody or antigen binding fragment thereof as a tool for human stem cell applications such as the selective marking, sorting or separation of human stem cell of the group comprising pluripotent stem cells, multipotent stem cells, oligopotent stem cells, unipotent cells, human germ cells, such as sperm cells, human progenitor cells of the group comprising early differentiated cell types derived from human stem cells, early differentiated cell types derived from human pluripotent stem cells (hPSC), undifferentiated human pluripotent stem cells (hPSC).

In one embodiment of the invention the sorting is made using Fluorescent Activated Cell sorting (FACS).

In one embodiment of the invention the sorting is made using magnetic beads.

In one embodiment of the invention the antibody or an antigen binding fragment thereof is used as a tumour marker for the diagnosis of malignant cancer tissue and in particular when the cancer tissue is selected from the group comprising carcinomas of the kidney, stomach, colon/rectum, larynx, lung, ovary, bladder and/or thyroid.

A fifth aspect of the invention relates to a hybridoma cell line that produces the antibody that has been deposited with the European Collection of Cell Cultures (Porton Down, Salisbury, SP4 OJG, United Kingdom) on 19 Nov. 2008, said hybridoma cell line having the ECACC accession number 08111901.

### DEFINITIONS

As used herein, “human pluripotent stem cells” (hPS) refers to cells that may be derived from any source and that are capable, under appropriate conditions, of producing human progeny of different cell types that are derivatives of all of the 3 germinal layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human embryonic stem (hES) cells, (see, e.g., Thomson et al. (1998), Heins eta. (2004), as well as induced pluripotent stem cells (see, e.g. Yu et al., (2007) Science 318:5858); Takahashi et al., (2007) Cell 131(5):861). The various methods and other embodiments described herein may require or utilise hPS cells from a variety of sources. For example, hPS cells suitable for use may be obtained from developing embryos. Additionally or alternatively, suitable hPS cells may be obtained from established cell lines and/or iPS (induced pluripotent stem cells).

As used herein, “feeder cells” refers to non-hPS cells that are co-cultured with hPS cells and provide support for the hPS cells. Support may include facilitating the growth and maintenance of the hPS cell culture by providing the hPS cell culture with one or more cell factors such that the hPS cells are maintained in a substantially undifferentiated state. Feeder cells may either have a different genome than the hPS cells or the same genome as the hPS cells and may originate from a non-primate species, such as mouse, or may be of primate origin, e.g., human. Examples of feeder cells may include cells having the phenotype of connective tissue such as murine fibroblast cells, human fibroblasts.

Without optimal culture conditions or genetic manipulation pluripotent stem cells will rapidly differentiate. Despite diverse genotypes and different techniques used for derivation and maintenance, all lines exhibit similar expression patterns for several markers, for example the markers for undifferentiated human pluripotent stem cells, such as the antigens SSEA3 and SSEA4, TRA-1-60, TRA-1-81, and Oct-4, ALP (alkaline phosphatase). Nevertheless, the lines are not identical: differences in expression of several lineage markers are known, and several imprinted genes show gene-dependent variations. Several sources of human pluripotent stem cells have been used in the present invention. However, in the present invention the term human blastocyst derived stem cells “hBS cells” is intended to encompass hPS cells derived from the cell lines SA001, SA002, SA002.5, AS034.1.1, SA121, SA167, SA461 (Cellartis, Göteborg, Sweden, www.cellartis.com) unless the use of a specific cell line is mentioned. The term human induced pluripotent stem cell (hiPS) cell is intended to mean pluripotent stem cells induced from adult human cells.

As used herein, the term “mEF cells” is intended to mean mouse embryonic fibroblasts.

As used herein the term “germlayer” is intended to mean any of the three major lineages known from developmental biology, i.e. ectoderm (giving rise to e.g. neural tissue), mesoderm (giving rise to e.g. connective tissue, cartilage, and bone) and endoderm (giving rise to tissues of the internal organs, such as the gut epithelium, liver and pancreas).

As used herein, the term “hFF cells” is intended to mean human foreskin fibroblasts. The hFF cells can further be commercially available or derived as described in the Methods part herein.

Basigin has been identified independently in several different model systems resulting in a long list of acronyms for this molecule including tumour collagenase stimulatory factor, EMMPRIN, neurothelin, OX-47, gp42, CE9, 5A11, HT7, M6, OK blood antigen, and most recently CD147. Basigin is the approved HUGO Gene Nomenclature Committee designation for the human gene and will be used to refer to the gene sequence and the expressed proteins in the present application.

Human basigin is expressed as at least two differentially spliced isoforms encoded by a single gene found on chromosome 19p13.3. The molecule is characterized by the presence of two extracellular immunoglobulin-like domains, a single transmembrane domain possessing a charged amino acid, and a short cytoplasmic tail containing a basolateral membrane-targeting motif. A more recently identified retina-specific isoform of basigin is distinguished by an additional immunoglobulin-like sequence in the extracellular domain of the protein. According to the current naming system of the National Center for Biotechnology Information, the larger retina-specific isoform has been renamed basigin-1 (accession number NM_{—}001728.2), and the prototypical isoform, possessing two immunoglobulin domains, has been renamed basigin-2 (accession number NM_{—}198589.1). Therefore as used herein the term “Basigin isoform 1” is intended to mean the longer isoform and the term “Basigin isoform 2” is the shorter isoform of Basigin respectively.

Markers for undifferentiated hPS cells: SSEA-3, SSEA-4 (stage specific embryonic antigen 3 and 4), TRA-1-60, TRA-1-81, Oct-4, ALP (alkaline phosphatase).

Marker for early differentiated hPS cells: SSEA-1

### BRIEF DESCRIPTION OF THE DRAWINGS

FIG. 1 shows a comparison between selected candidate and commercially available pluripotency markers, i.e. Oct-4, SSEA-4 and TRA 1-81 using immunofluorescent analysis as stainings on human blastocyst derived stem cells (hESC).

FIG. 2 (a-b) shows the staining of live cells in suspension on human blastocyst derived stem cells (hESC).

FIG. 3 shows the amino acid sequence of the Basign isoform 1 (seq. 1) vs. the Basigin isoform 2 (seq. 2).

FIG. 4 shows HES5:3:3 mAb antigen detection by A) Western Blot B) Native western blot and C) Native western blot with more protein loaded.

FIG. 5 shows the identification of Basigin isoform 1.

FIG. 6 shows morphological and immunohistochemical characterization of a hiPS cell line cultured on a mEF feeder layer.

FIG. 7 shows immunofluoroscence stainings of undifferentiated hiPS cells after thaw on hUWIL cells using HES5:3:3 and other pluripotent cell markers.

FIG. 8 shows immunohistochemistry stainings on cancer tissue sections using HES5:3:3.

FIG. 9 shows the DNA fragments covering the unique segment of Basigin isoform 1 inserted into the vector f44-8.

FIG. 10 shows the extra 115 amino acids of Basigin isoform 1 and the overlapping fragments, BSG F1-BSG F4, displayed on phage.

FIG. 11 demonstrates the Phage ELISA specific reactivity of MAb HES5:3:3 against the fragment BSG F3

### DETAILED DESCRIPTION OF PREFERRED EMBODIMENTS

In the following examples the invention will be described in more detail. However, the described embodiments mentioned below are only given as examples and should not be limiting to the present invention. Other solutions, uses, objectives, and functions within the scope of the invention as claimed in the below described patent claims should be apparent for the person skilled in the art.

### Example 1

### Preparation of the Antibody

hBS Cell Lines Used and their Culture

All human blastocyst derived stem (hBS) cell lines used in this study (SA001, SA002, SA002.5, AS034.1.1, SA121, SA167, SA461; Cellartis, Göteborg, Sweden, www.cellartis.com) were derived and cultured as described previously [Heins N, Englund MCO, Sjöblom C et al. Derivation, characterization, and differentiation of human embryonic stem cells. STEM CELLS 2004; 22:367-76]. In brief, hBS cells were maintained on a layer of Mitomycin C (Sigma-Aldrich, Stockholm, Sweden; http://www.sigmaaldrich.com) inactivated mouse embryonic fibroblasts in VitroHES™ medium (Vitrolife AB; Kungsbacka, Sweden; http://www.vitrolife.com) supplemented with 4 ng/ml human recombinant basic fibroblast growth factor (hrbFGF) (Gibco/Invitrogen corporation, Paisley, UK; http://www.invitrogen.com). The hBS cell colonies were routinely passaged every 4-5 days by mechanical micro-dissection into small pieces and subsequently transferred to freshly prepared culture dishes.

hBS cells used in screening and magnetic separation experiments were cultured on a layer of Mitomycin C inactivated human foreskin fibroblasts (hFF) (CRL-2429, American Type Culture Collection (ATCC), Manassas, Va.; http://www.atcc.org) and enzymatically passaged to a single cell suspension by using TrypLE Select (Invitrogen) as previously described [Ellerström C, Strehl R, Noaksson K, Hyllner J, Semb H. “Facilitated expansion of human embryonic stem cells by single cell enzymatic dissociation.” Stem Cells. 2007 Mar. 22]. Routine characterization of hBS cell cultures was carried out according to the methods described in Heins et al., 2004 [ref].

### Preparation of Immunization Antigen

Undifferentiated hBS cells from the hBS cell line SA167 was used for the immunization of mice. Prior to the antigen preparation, the hBS cells were transferred to feeder-free culture in Matrigel™-coated dishes and maintained under feeder-free conditions for 10 passages as described by Sjögren-Jansson et al 2005 [Sjögren-Janson E, Zetterström M, Moya K et al. Large-Scale propagation of four undifferentiated human embryonic stem cell lines in a feeder-free culture system. Dev Dyn. 2005; 233:1304-1314]. hBS colonies were dissociated by using 0.5 mM EDTA in 1× phosphate buffered saline (PBS, GIBCO/Invitrogen), washed twice in PBS to remove traces of EDTA and finally resuspended in PBS. Whole cell antigen mixture from human feeder cells (hFF, CRL-2429, ATCC) was prepared in a similar way.

### Production of Hybridoma Cells

Conventional hybridoma technology [Köhler G, Milstein C. Continuous cultures of fused cells secreting antibody of predefined specificity. Nature. 1975 Aug. 7; 256(5517):495-] was used to establish hybridoma cell lines producing mAbs against hBS cells. Three Balb/c mice were immunized and a total of three fusions were performed 2, 9 and 12 months post-immunization. Briefly, 8 week old Balb/c female mice were immunized intraperitoneally with 100 μl crude whole cell antigen mixture containing approximately 2×10⁶ whole undifferentiated hBS cells (SA167, Cellartis AB, prepared as described above) together with 100 μl of adjuvans Ribi (Corixa, now GlaxoSmithKlein, GSK). Booster immunizations were performed at regular intervals, ending with a pre-fusion booster three days before fusion. The animals were sacrificed with CO₂ and the spleens were surgically removed. B-cells from the immunized mice's spleen were fused with the myeloma cell line P3X63Ag8.653 (CRL-1580, purchased from American Type Culture Collection (ATCC), Manassas, Va.; http://www.atcc.org) and hybridomas were cultured in 96-well plates for three weeks (Hypoxanthine Aminopterin Thymidine (HAT) component described in Köhler G, Milstein C, 1975) in selection medium promoting selective growth of the hybridoma cell lines while both non-fused B-cells and non-fused myeloma cells die off. Medium samples (supernatants) containing secreted antibodies were collected from each well containing hybridoma cells and formed the basis for the subsequent immunocytochemical screening and selection of hybridoma cell lines producing antibodies of the desired specificity.

A similar protocol was used to establish hybridoma cell lines producing mAbs against antigens from hFF feeder cells. One mouse was immunized with whole cell antigen mixture containing approximately 0.3×10⁶ hFF cells and the fusion was performed seven months after the first immunization.

### Primary Screening

To allow efficient screening of a large number of hybridoma supernatants, the primary screening system was designed to contain two different cell types simultaneously, i.e. hBS cells to allow positive screening of potential candidates as well as hFFs to eliminate candidates with low hBS cell specificity. To prepare screening plates for primary screening, hBS cell colonies were dissociated from the feeder cells with TrypLE Select (Gibco/Invitrogen) for approximately 5-10 minutes. The single cell suspension of hBS cells was seeded into 96-well plates (Nalge Nunc International; Rochester, N.Y.; http://www.nalgenunc.com) containing a layer of mitotically inactivated hFFs in VitroHES™ medium supplemented with hrbFGF. Plates were incubated and culture medium was renewed every other day for 7-8 days until colonies of undifferentiated hBS cells had formed and the cells in the plates were fixed in 4% Paraformaldehyde (PFA, Histolab Products AB, Västra Frölunda, Sweden; http://www.histolab.se) solution for 10 minutes at RT. Screening plates containing hFF cells were prepared in a similar way as described for the hBS cells. Screening was thereafter performed immunocytochemically by using either fluorescein isothiocyanate (FITC)—conjugated or horseradish peroxidase (HRP)—conjugated secondary antibodies.

### Secondary Screening

After elimination of all non-reacting candidates and all candidates with insufficient specificity to undifferentiated hBS cells, secondary screenings of remaining potential candidates was performed on a number of different cell types. The purpose of the secondary screening was to eliminate candidates showing specificity to differentiating hBS cells but also to adult human tissue cells. Plates of differentiated hBS cells were prepared by leaving hBS cells to spontaneously differentiate in VitroHES™ medium for around 20 days (SA034.1 21 days, SA002.5 19 days, SA121 21 days) while culture medium was renewed every 2-3 day.

Plates of human embryonic stem cell derived mesenchymal progenitors (hBS-MPs) were prepared by seeding hBS-MP cells into 96-well plates at a density of 80000 cells/ml (45000/cm²) in hBS-MP medium consisting of DMEM (high glucose with glutamax, without pyruvate) supplemented with 10% fetal bovine serum (FBS), 1% PEST and 10 ng/ml human recombinant basic fibroblast growth factor (hrbFGF) (All from Gibco/Invitrogen). The cells were cultured for 5 days before fixation.

To exclude cross-reaction with highly differentiated tissue, some selected supernatants were tested on adult human tissue slides (human frozen tissue panel slides, cat. No. T6234431; Biochain; Hayward, Calif.; http://www.biochain.com).

### Cloning of Hybridomas

The hybridoma cells that were selected through the different stages of screening (primary and secondary) were subsequently cloned by a single-step procedure under visual control. This was done to ensure that each hybridoma culture contained only one hybridoma clone of a defined specificity. Briefly, the hybridoma cells were diluted in medium to a concentration of 10⁴ cells/ml. Small drops of the cell suspension (0.4-0.5 μl) were placed on the bottom of 96-well plates and examined microscopically. Hybridoma culture medium was added only to the wells that had been confirmed to contain one single cell. Only 8 wells were processed simultaneously to avoid evaporation of the culture medium. This procedure secures that hybridoma growth initiates clonally from one cell. Cloning resulted in several subclones, these were screened as described for hybridomas above. The best clone, proven to be good for the purposes intended for the invention, was recloned in order to ensure monoclonality. From the final clone, cell banks were established to secure future antibody production from the hybridoma. The hybridoma HES5:3:3 was deposited according to The Budapest Treaty of 1977 with the European Collection of Cell Cultures (Porton Down, Salisbury, SP4 OJG, United Kingdom) on 19 Nov. 2008, with ECACC accession number 08111901.

### Determination of Antibody Isotype and Concentration

Determination of isotopes (IgG1, IgG2a, IgG2b, IgG3, IgM, IgA) of the antibodies obtained from the selected hybridoma clones was preformed in an ELISA assay. Briefly, antibodies in medium supernatants were captured in maxisorp 96-well plates (Nunc, Roskilde, Denmark) coated with polyclonal rabbit-anti-mouse IgG or IgG+A+M (Zymed, South San Francisco, Calif., USA). Antibodies were then traced using isotype specific rabbit-anti-mouse HRP-conjugated polyclonal antibodies (Zymed). The mAb concentration in supernatants from cloned hybridomas was determined by an ELISA assay. Antibodies were captured in 96-well maxisorp-plates (Nunc) coated with polyclonal affini-pure goat anti-mouse IgG+IgM (H+L) (Jackson Immuno Research, West Grove, Pa., USA). The mAbs were traced using polyclonal rabbit anti-mouse Immunoglobulins/HRP (Dako, Glostrup, Denmark). The results were determined using standard curves established with mAbs with known isotypes and concentrations.

Purification and Biotinylation of Selected mAbs

Further in vitro expansion was performed from hybridoma clones in established cell banks. Cultivation were done by inoculation of 2-5×10⁴ cells/mL in DMEM (Sigma), 5%

Fetal Calf Serum (Sigma or Hyclone) and supplement containing 36 mg/l L-Aspargine, 116 mg/l L-Arginine-HCl, 10 mg/l Folic acid, 292 mg/l L-Glutamine and 110 mg/l Na-Pyruvate, in roller bottles. Cultures were allowed to grow for three weeks. Monoclonal antibodies were purified from the culture medium by ProSep-vA (Millipore) affinity chromatography, according to the manufacturer's instructions. The eluted antibody was dialysed and concentrated in 0.15 M NaCl. Concentration of purified antibody was calculated from absorbance at 280 nm. Antibody stock solutions were stored in −70° C.

Biotinylation of purified antibodies was carried out in 1 mol/l carbonate buffer pH 8.5 with 5 times of molar excess of biotinamido-caproate N-hydroxysuccinimide ester (Sigma) for 2 h at room temperature. Non-reacted reagents were removed by gel filtration on PD-10 columns (Amersham Bioscience) in 50 mM Tris-HCl pH 7.75.

### Species Specificity Testing

In order to determine whether the selected antibodies were specific to human cells, mouse cells or both, the species specificity of the selected antibodies was determined in mouse embryonic stem (mES) cells (mES line R1 in passage 24). The cells were cultured both with and without mitotically inactivated mouse embryonic fibroblast (mEF) feeders and in the presence of Cytokine leukemia inhibitory factor (LIF). The mES cells were fixed and stained with selected mAbs from the screening process together with two positive controls specific for Oct-4 and SS EA-1 (Santa Cruz Biotechnology, Santa Cruz, Calif., http://www.scbt.com) as well as a negative control.

To determine the specie-specificity of the selected candidates, mouse embryonic stem (mES) cells were employed. In immunofluorescent analysis HES5:3:3 stained exclusively hBS cells and thus can be considered as specie-specific.

### Example 2

### Characterization of Antibody Reactivity

Monoclonal antibodies are widely used for the analysis of cells in-vitro but can furthermore be applied to quantify and select certain cell populations in a mixed culture. Oct-4 and Nanog antibodies are commonly used for characterization of human BS cells and indeed they are good and specific nuclear antibodies for the purpose. The antibody specificity of the selected clone HES5:3:3 was tested by letting the antibodies react with undifferentiated hBS cells or undifferentiated hBS cells mixed with feeder cells and comparing their reactivity to the commonly used antibodies.

The basic immunocytochemical procedure included fixation of the undifferentiated hBS cells or undifferentiated hBS cells mixed with feeder cells in 4% PFA (Histolab) for 10 minutes, permeabilization of the test material using 0.5% Triton X-100 solution (Sigma-Aldrich), subsequent blocking of unspecific binding sites with 5% FBS in 1×PBS (Invitrogen) and incubation of the samples with diluted hybridoma supernatants or primary antibody solution over night at 4° C. For visualization of bound primary antibody, samples were incubated with a diluted fluorescein isothiocyanate—(FITC-) or horseradish peroxidase—(HRP-) conjugated secondary antibody (Goat anti-mouse IgG, H+L). Peroxidase, if used, was visualized by AEC resulting in an insoluble red-brown reaction product.

In all test systems, at least one positive control was included. For hBS cell cultures the positive control was SSEA-4 (Stage-Specific Embryonic Antigen-4) that is a marker included in the common hBS cell characterization panel.

The results from the present study indicate that the HES5:3:3 is more specific to undifferentiated human BS cells because the Oct-4 and Nanog antibodies cross-react with early differentiating human BS cells in a manner that is not detected using the HES5:3:3 antibody. The HES5:3 is a cell surface antibody while the Oct-4 and Nanog antibodies are nuclear antibodies.

Competitive Binding Studies with Commercially Available Antibodies for Undifferentiated hBSC

The affinity of the purified top candidate to its antigen was tested in an antibody dilution and competitive binding study with commercially available antibodies. In the dilution experiment hBS cells were incubated over night with the selected novel mAb HES5:3:3 in a range of concentrations (0.1-250 μg/ml). After washing, a second incubation with the biotin-conjugated version of the same selected mAb at a concentration of 1 μg/ml was carried out, followed by a streptavidin-FITC secondary antibody. In the competition experiment the ability of the top candidates to block the binding of commercial antibodies was tested and the procedure of the dilution experiment was repeated using commercially available antibodies against TRA-1-60, TRA-1-81 and SSEA-4 (Santa Cruz) instead of the biotin-conjugated mAb in the second step. As a secondary antibody a fluorescein isothiocyanate—(FITC) conjugated version was used in incubations for 60 minutes at room temperature (Jackson Immunoresearch Laboratories, West Grove, Pa., http://www.jacksonimmuno.com).

The binding of HES5:3:3 was unchanged down to 0.5 μg/ml and a slight decrease in intensity was shown at 0.2 μg/ml. A significant drop in the signal was observed at 0.04 μg/ml.

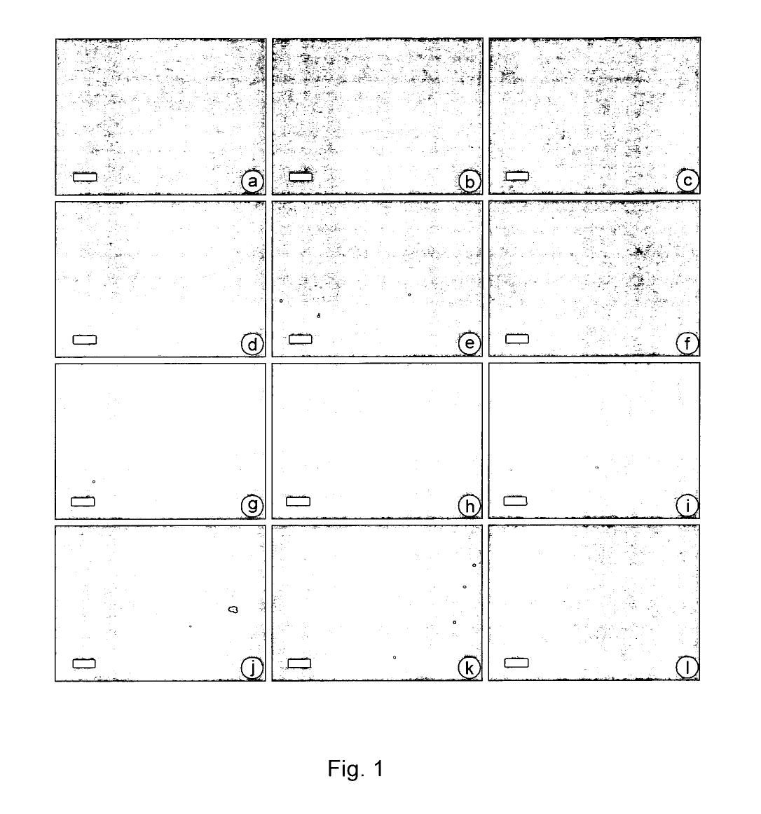

Comparison between the selected candidate and commercially available pluripotency markers, i.e. antibodies specific to Oct-4, SSEA-4 and TRA 1-81 was made by immunofluorescent analysis. Simultaneous incubation of hBS cells with Oct-4 and HES5:3:3 antibodies imply that HES5:3:3 is a better pluripotency marker based on the observations that HES5:3:3 doesn't stain migrating individual cells with a higher nucleo cytoplasmic ratio, commonly referred as cells in differentiation, whereas antibodies directed to Oct-4 do (FIG. 1 a-c). In addition, HES5:3:3 did not show any positive reaction against feeder cells (FIG. 1f), while commonly used TRA 1-81 did (FIG. 1e).

Direct Conjugation of mABs with Fluorescent Dyes for Use in Immunocytochemistry

The selected, cloned and purified mAbs were labelled with a fluorescent dye by using a Zenon mouse specific isotype IgG labeling kit (mouse IgG Alexa Fluor 594 and mouse IgG Alexa Fluor 488; Invitrogen). The labelling was performed following the instructions from the manufacturer. Briefly, the appropriate dilution of antibody solution was prepared in PBS, 5 μl of the labelling reagent, which is the amount required for labelling 1 μg of the monoclonal antibody, was then added and the mixture was incubated for 5 minutes at room temperature. During that time, the labelling reagent reacts forming a labelling complex between the antibody and the fluorescent dye. The labelling reagent contains a fluorophore labelled with the Fab specific anti-IgG fragment designed against to the Fc portion of the primary antibody. The Fab in excess was taken up by adding 5 μl of the blocking reagent to the mixture.

For immunocytochemistry, cell samples from several different hBS cell lines, such as SA002, SA167, SA461 (Cellartis AB), were fixed by adding 4% PFA at room temperature for 10 minutes. After washing, nonspecific binding sites were blocked by incubation with 10% FBS in PBS (both from Gibco/Invitrogen) for 30 minutes. The conjugated fluorescent antibody was then applied to the samples for 30-60 minutes at room temperature, and protected from light during the incubation. When the mAb staining was complete the cell nuclei were stained with 4′,6-diamidino-2-phenylindole (DAPI) (Sigma-Aldrich). The samples were mounted with fluorescent mounting media (DAKO; http://www.dako.com) and examined under a fluorescence microscope.

### Staining of Live Cells in Suspension

Undifferentiated hBS cells cultured on top of mitotically inactivated hFFs were removed from the culture system by enzymatic treatment. A proteolytic enzyme, TrypLE Select (containing 1 mM EDTA; TrypLE Select; Invitrogen), was added in order to get a single cell suspension by breaking the bonds that attaches the hBS cells to the hFFs monolayer and to each other. Traces of the enzyme were removed and discarded after centrifugation, and the pellet was resuspended in 5% FBS in PBS. A mixture of undifferentiated hBS cells and hFFs were obtained and incubated for 30 minutes at +4° C. with direct-conjugated versions of selected monoclonal antibodies (fluorescence conjugation protocol previously described above). PBS was added followed by centrifugation for 5 minutes at 4° C. Supernatant was discarded, and the cell pellet was resuspended in 5% FBS in PBS. From the cell suspension a small amount of cells were transferred to a slide, (cover by a coverslip), and then inspected in an inverted fluorescence microscope.

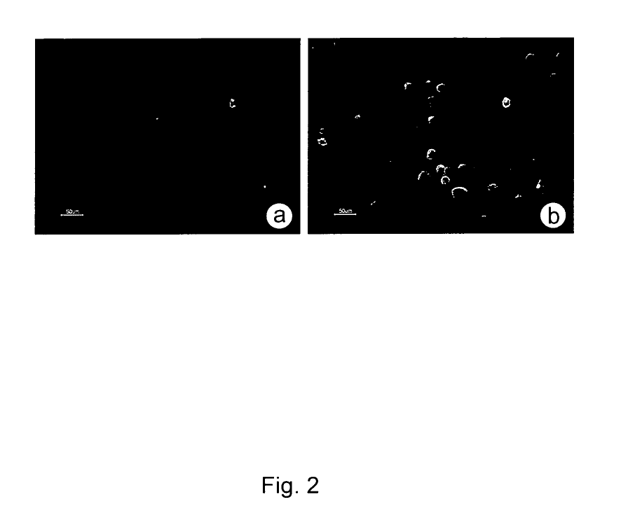

The mAb HES5:3:3 was clearly reacting with cells in suspension (FIG. 2 a-b) and therefore able to bind to an antigen located on the surface of undifferentiated hBS cells.

### Example 3

### Identification of the Antigen Reacting with the Selected Antibody Candidates

To determine the antigen to the selected antibody, the protein was first determined by western blot as described below, furthermore the protein was purified (isolated) and migrated in a SDS PAGE and the identity of the purified protein was determined with FT mass spectrometry analysis

Human ESGs (SA461) used in this study were provided by Cellartis AB (Göteborg, Sweden, www.cellartis.com). Feeder free hBS cells were washed three times with PBS and suspended in 10 mM tris-HCl, pH 7.4, 10 mM MgCl₂, 10 mM KCl, 1 mM DTT, 1 mM mM phenylmethyl sulphonyl fluoride, 10 μg/ml aprotinin, 1 μg/ml leupeptin and pepstatin. The cells were lysed by addition of Triton X-100 (to a final concentration of 1%) and sonicated for 5×3 seconds. NaCl was added to a final concentration of 0.2M. The cell lysate was incubated on ice for 1 h and clarified by centrifugation for 1 h at 13000×g. Collected supernatant was used in subsequent enrichment and analysis of the antigen.

Native Western blot was employed in order to characterize the mAbs. The proteins from the cell lysate above were separated on 6% polyacrylamide gels in the absence of sodium dodecyl sulphate (SDS) and dithiothreitol (DTT). After electrophoresis, proteins were detected immunologically following semi-dry electrotransfer onto polyvinylidene difluoride (PVDF) blotting membranes [Immobilon-P; Millipore] for 1 h 40 minutes, 110 mA/gel in transfer buffer (48 mM Tris, 39 mM Glycin, 10% MeOH). The blots were blocked with 5% non-fat dry milk in PBST (phosphate-buffered saline and 0.1% Tween 20) for 1 h and then incubated with primary antibody in blocking solution overnight, 4° C. After washing with PBST, the membrane was incubated with the AP Conjugated Goat Anti-Mouse IgM+IgG+IgA(H+L) secondary antibody (Southern Biotechnology Associates, Inc) in blocking solution for 1 h at room temperature. For detection, AP-develop (50 mM Tris pH 9, 5 mM MgCl₂) with addition of BCIP and NBT [Promega] were used.

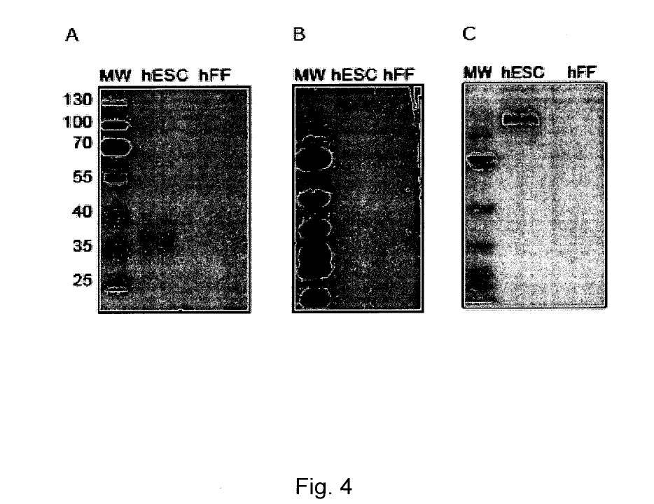

For SDS PAGE the proteins were separated on 6% polyacrylamide gels in the presence of SDS and DTT. Gels were stained with Sypro®Ruby Protein Gel Stain (BioRad) and protein bands were visualized under UV light. Based on molecular weight markers, the HES5:3:3 mAb detected a very diffuse antigen band of about 40 kDa (FIG. 4A). Therefore, it was hypothesized that this antibody recognizes the antigen in its native form. To test this, the hBS cell protein extract was electrophoresed on polyacrylamide native gels and probed with mAb HES5:3:3. The proteins were separated on 6% polyacrylamide gels in the absence of SDS and DTT.

As can be seen in FIG. 4 the antigen band was successfully detected (FIG. 4B shows native Western blot and 4C a native Western blot with more protein loaded). In light of these findings it was concluded that HES5:3:3 mAb recognizes the antigen under native conditions and can be recommended for use in native western blot. No band was detected with extract from human fibroblast feeder cells (hFF).

To identify the target antigen of HES5:3:3 mAb on hBS cells, enrichment of the antigen under native conditions was performed. Whole cell lysate was incubated with covalently linked HES5:3:3 mAb to Protein G magnetic resin (Dynabeads® Protein G, Invitrogen). The monoclonal antibody HES5:3:3 was coupled by crosslinking to magnetic Dynabeads® Protein G according to manufacturer's protocol (Invitrogen) at a ratio of 10 μg antibody to 20 μl of beads. The hBS cell extract was incubated with the antibody-linked beads overnight at 4° C. The beads were washed three times with 500 μl of ice-cold PBS. Two elution steps were performed; the first with the elution buffer (50 mM Tris-Hcl pH 7.5 1 M Nacl, 0.1% NP-40 and 1 mM DTT) and second with 0.1 M Citric acid. The eluted fraction was divided for analysis on native PAGE, native Western blot and SDS PAGE.

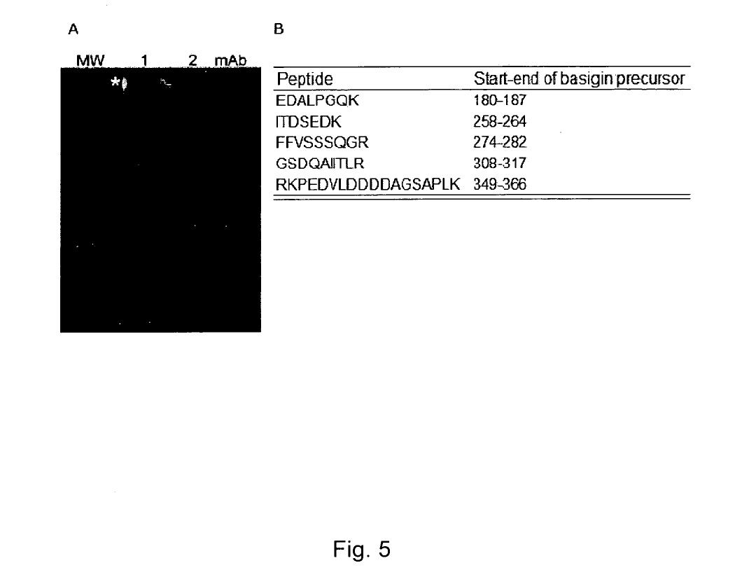

Proteins that were captured by affinity interactions were resolved on native protein gels and probed with HES5:3:3 mAb. This can be seen in FIG. 5A wherein enriched antigen from hBS cell lysate has been eluted with NaCl (lane 1) and citric acid (lane 2), resolved on native gel and stained with Sypro®Ruby. The corresponding band on a Sypro®Ruby stained gel (protein band marked with asterix) was excised and identified with nano-LC FT-ICR mass spectrometry analysis (FIG. 5B) to be Basigin isoform 1. From protein database search with peptides obtained, the antigen band was identified as Basigin isoform 1 (BASI, accession No P35613) (FIG. 5B) with molecular weight of 42 kDa.

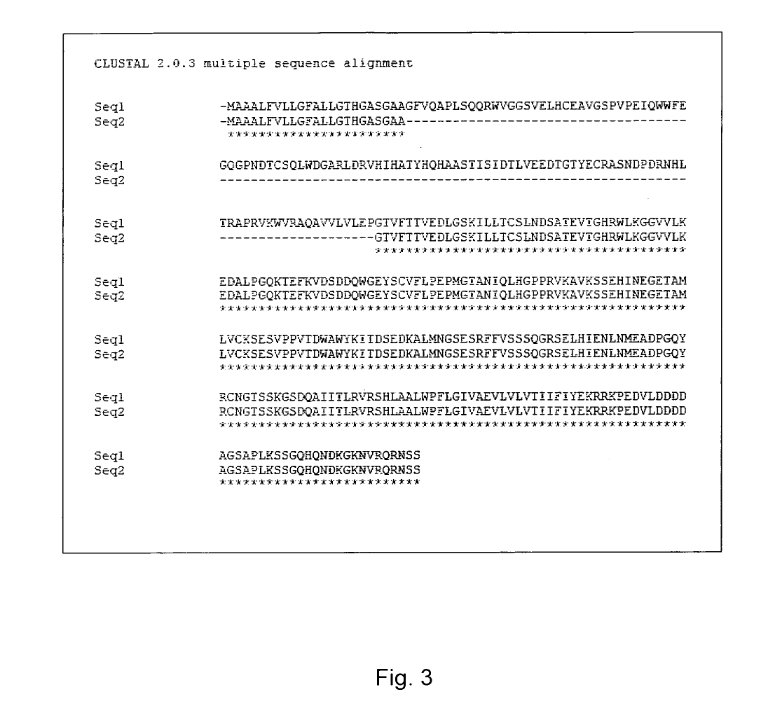

Interestingly, the predicted molecular weight of Basigin isoform 1 corresponds well with detected bands on western blot. Therefore and in order to validate results, immunoprecipitation with HES5:3:3 mAb was repeated and the eluate was resolved on SDS protein gel The band corresponding to 40 kDa was isolated and identified with nano-LC FT-ICR to be Basigin isoform 1. This protein is localized to the cell membrane and plays pivotal roles in cancers and embryonic implantation. The Basigin isoform 1 has been isolated and sequenced and in FIG. 3 the Basigin isoform 1 sequence (seq 1) is aligned with the Basigin isoform 2 sequence (seq 2) for comparison. Isoform 1 is 115 amino acids larger than Basigin isoform 2.

The following amino acid sequence is the complete Basigin isoform 1 sequence:

(SEQ ID NO: 1)
MAAALFVLLG FALLGTHGAS GAAGFVQAPL SQQRWVGGSV
ELHCEAVGSP VPEIQWWFEG QGPNDTCSQL WDGARLDRVH
IHATYHQHAA STISIDTLVE EDTGTYECRA SNDPDRNHLT
RAPRVKWVRA QAVVLVLEPG TVFTTVEDLG SKILLTCSLN
DSATEVTGHR WLKGGVVLKE DALPGQKTEF KVDSDDQWGE
YSCVFLPEPM GTANIQLHGP PRVKAVKSSE HINEGETAML
VCKSESVPPV TDWAWYKITD SEDKALMNGS ESRFFVSSSQ
GRSELHIENL NMEADPGQYR CNGTSSKGSD QAIITLRVRS
HLAALWPFLG IVAEVLVLVT IIFIYEKRRK PEDVLDDDDA
GSAPLKSSGQ HQNDKGKNVR QRNSS

It is further contemplated that HES5:3:3 may specifically bind to carbohydrates present on the Basigin isoform 1 protein.

### Specificity Comparison Between HES5:3:3 and Commercially Available Antibodies for Basigin/Emmpirin

HES5:3:3 is a monoclonal antibody, specific for undifferentiated human pluripotent stem (hPS) cells. It shows specificity for the Basigin isoform 1, a member of the immunoglobulin superfamily that is also known as EMMPIRIN, short for extracellular matrix metalloproteinase inducer, and recently has been designated CD147 (cluster of differentiation 147). As members of the immunoglobulin superfamily, basigin plays fundamental roles in intercellular recognition involved in various immunologic phenomena, differentiation, and development. Basigin is thought also to play a role in intercellular recognition.

Commercially available antibodies for Emmpirin, specific for the N- and C-terminal, N-19 and C-19 respectively (Santa Cruz Biotechnology Inc., CA, USA) respectively, does not show the same specificity. These commercially available antibodies also stain the mouse embryonic feeder cells and generate an unsatisfying fluorescence background.

The HES5:3:3 generates a very specific staining pattern on undifferentiated hPS cells, clearly visualising the membranes, even at extremely low concentrations (0.25 μg/ml versus 4 μg/ml for the Santa Cruz's antibodies). This is not the case for the previously available antibodies (N-19 and C-19) compared here.

TABLE 1
Specificity comparison between HES5:3:3 and commercially
available antibodies for Basigin/Emmpirin

| | HES5:3:3 | N-19 | C-19 |
|---|---|---|---|
| Dilution | 0.25 μg/ml | 4 μg/ml (2 μg/ml) | 4 μg/ml |
| Specificity: | mouse IgG | goat IgG polyclonal | goat IgG |
| | monoclonal | | polyclonal |
| mEF stained | no | background | background |
| Extra-embryonic | Yes | background | background |
| endoderm stained | | | |

### Example 4

### Separation Experiments

The antibodies reacting with surface antigens on the cells, such as the ones developed in this study, can be used to label and select living cells using FACS or magnetic bead based sorting. These selection procedures will be useful to enrich populations of undifferentiated hBS cells in cultures showing areas of unwanted differentiation or from feeder supported cultures. Alternatively, remaining undifferentiated hBS cells can be depleted from differentiated cell populations for transplantation experiments to avoid formation of teratomas in the recipients. Thus, HES5:3:3 can be used in these very important technologies whilst there is very limited use of the OCT-4 and Nanog antibodies in such applications.

### Flow Cytometry

Colonies of undifferentiated hBS cells (line SA002, Cellartis AB) cultured on a supporting feeder layer were treated with TrypLE Select (Invitrogen) to obtain a single cell suspension. Human embryonic stem cell derived mesenchymal progenitors (hES-MP) cells were expanded in monolayer to 80% confluence, and thereafter harvested using 0.05% trypsin-0.53 mM EDTA. The cells were stained with the established cell surface marker (HES5:3:3). As a negative control, hES-MP cells derived from the hBS cell line SA002 as well as isotype specific control antibodies were used. At least 10.000 events were acquired for each sample using the Fluorescent-activated Cell Sorting (FACS) Aria flow cytometer and the FACSDiVa software (Becton Dickinson). The 488 nm argon ion laser was used to excite samples, with emission being measured using appropriate band pass filters. The cells were acquired and gated by forward (FSC) and side scatter (SSC) to exclude debris and cell aggregates. Dead cells were excluded by gating on FSC and SSC.

### Magnetic Separation Employing HES5:3:3,

To prove the use of the HES5:3:3 antibody for separation of hBSC from feeders (hFF and mEF) magnetic cell separation was performed according to the manufacturer's manual (Miltenyi Biotec, Bergisch Gladbach, Germany http://www.milteniybiotec.com). Briefly, mixtures of cells (hBS cells and hFF cells or hBS cells and mEF cells), were dissociated into single-cell suspension using TrypLE™ Select and washed once by adding cold buffer (PBS, 2% FBS and 1 mM EDTA) and centrifuged for 5 minutes at 400 g. The Cells were incubated with the primary antibody HES5:3:3 at a concentration of 1-2 μg/ml for 20 minutes at +4° C. To remove unbound primary antibody, cold buffer was added and cells were centrifuged again. GoatAnti-Mouse IgG MicroBeads (Miltenyi Biotec) were added to the single cell suspension to a final concentration of 20-25% and cells were incubated for 15 minutes at +4° C. To remove unbound secondary antibody, cold buffer was added and cells were centrifuged. A MS column (Miltenyi Biotec) was rinsed and the cell suspension (cells with bound primary and secondary antibodies) was applied to the column. All cells passing through the column together with the cells removed in the three following washing steps were denoted the negative (i.e. cells that are not recognized by the antibody and have not received a magnetic label, plus unbound antibodies) fraction. The column was removed from the separator and the positive fraction (i.e. mAb recognized cells) was flushed out with cold buffer. Immediately after separation the fractions were centrifuged and fixed in 4% PFA.

The cell separation was evaluated immunocytochemically. The employed antibodies were labelled using the Alexa Fluor 488 Monoclonal Antibody Labeling Kit or the Alexa Fluor 594 Monoclonal Antibody Labeling Kit as described previously. An unseparated cell suspension was stained by incubation with the primary antibody HES5:3:3 at a final concentration of 1 μg/ml and DAPI for 2 hours at RT. The positive and negative fractions from separation with HES5:3:3 were stained with fluorescently labelled HES5:3:3 (1 μg/ml), DAPI and FITC—conjugated goat Anti mouse IgG (2.5 μg/ml). To remove unbound antibody, PBS was added and cells were centrifuged. The cells were then mounted with fluorescent mounting media (DAKO; Glostrup, Denmark, http://www.dako.com) and samples were examined under a fluorescent microscope. The results were evaluated using the software ImageJ as a counting tool.

In order to further test the performance of the selected antibody, magnetic separation was employed to separate SCED (Single Cell Enzymatically Dissociated)-cultured hBS cells from their feeder cells (hFFs). A single cell suspension consisting of a mixture of undifferentiated hBS cells and hFFs were stained with the magnetically labelled antibody HES5:3:3 (see procedure above). The separation efficiency was evaluated through staining of both positive as well as negative fractions by using fluorochrome labelled secondary antibodies whereby different cell types could be counted. The results indicate a high specific affinity HES5:3:3 the purity for the selection of hBS cells was 98.4% (σ 1.3 (n=5)) when hBSCs and hFF cells were present in the cell suspension and 97.5% (σ 3.5 (n=2)) in a mixture of hBSCs and mEF cells.

### Example 5

### Effects of Basigin in hBSC Culture for Enhanced Survival and Adhesion

Effects of Basigin on Survival of Dissociated hBSC Following Enzymatic Digestion.

Following enzymatic digestion to single cells, hBSC undergo an apoptotic process, resulting in the loss of a high proportion of cells. The purpose of this experiment is to assess whether the presence of basigin may have any influence to prevent the initiation of the apoptotic cascade, or to rescue cells following dissociation. In order to assess the potential of basigin to prevent the enzymatic dissociation induced apoptosis of hBSC, SA461 and SA121 hBS cells were digested using trypsin, passed through a 100 μM filter and seeded in vitroHES™ onto 96 well plates with basigin either alone or in combination with other commonly used pre-coated matrix components including fibronectin, collagen, laminin, matrigel, and vitronectin. The influence of basigin was assessed both as a coating substrate for the culture vessel and as a media supplement.

Seeding was performed at a density of 10,000 cells per well of a 96 well plate, attached cells were fixed 12 hours later using parafomaldehyde and stained using DAPI. Relative cell density was then assessed using either a fluorescent microscope or omega plate reader. The result showed an increase in cell survival associated with the presence of basigin, either as a culture vessel coating, or as a media constituent.

Effects of Basigin on Attachment of Dissociated hBSC Following Enzymatic Digestion.

The objective of this experiment is to determine whether basigin has any function in promoting the attachment of hBS cells following enzymatic passaging. We are expecting to demonstrate an increased ability of hBS cells to reattach to culture vessels post enzymatic dissociation.

By modifying the basic protocol described above, SA461 and SA121 hBS cells were briefly enzymatically digested, scraped, triturated to small clusters and seeded in vitroHES™ media onto 96 well plates with basigin either alone or in combination with other commonly used pre-coated matrix components including fibronectin, collagen, laminin, matrigel, and vitronectin. The influence of basigin was assessed both as a coating substrate for the culture vessel and as a media supplement.

Seeding was performed at a density of 10,000 cells per well of a 96 well plate, attached cells were fixed 12 hours later using parafomaldehyde and stained using DAPI. Relative cell density was then assessed using either a fluorescent microscope or omega plate reader.

The results showed higher cell densities in wells containing basigin, either as a culture substrate or as a media component.

Effects of Basigin on hBSC Phenotype in Supportive Culture.

The purpose of this experiment is to determine whether the inclusion of basigin in culture conditions supportive of undifferentiated hBSC expansion is sufficient to trigger the differentiation of the hBS cells. As such it is an assessment of the likely contribution of basigin to the differentiation process.

In order to assess the potential of basigin to initiate differentiation, SA461 and SA121 hBS cells were plated from feeder free maintenance culture into fully supportive culture conditions. Basigin was included in the media at a concentration of 100 ng/ml. Cells were maintained using routine feeder free maintenance protocols for 10 days then expression levels of markers of pluripotency and differentiation checked by RT-qPCR. These markers include, but are not limited to NANOG, OCT4, SOX2, EOMES, brachyury, SOX17, CDX2 and FOXA2. The result showed that the expression levels of these marker genes as assayed by qPCR are not changed significantly following exposure of the cells to exogenous basigin.

Effects of Basigin on hBSC Phenotype in Non Supportive Culture.

The purpose of this experiment is to determine whether the inclusion of basigin in culture conditions non-supportive of undifferentiated hBSC expansion is sufficient to prolong the expression of markers of the undifferentiated phenotype. As such it is an assessment of the likely contribution of basigin to the maintenance of the undifferentiated phenotype.

In order to assess the potential of basigin for it's contribution to the maintenance of the expression of pluripotency markers and the absence of induction of differentiation markers (to include but not be limited to NANOG, OCT4, SOX2, EOMES, brachyury, SOX17, CDX2 and FOXA2). SA461 and SA121 hBS cells were plated from feeder free maintenance culture into serum containing media on a gelatin matrix, non-supportive for hBSC self renewal, and basigin was included in the media at a concentration of 100 ng/ml. Cells were maintained using routine maintenance protocols until the onset of morphological change and samples collected to assay expression levels of markers of pluripotency and differentiation by RT-qPCR every 48 hours. The results demonstrated delayed downregulation of pluripotency markers (NANOG, OCT4, SOX2) and the delayed induction of differentiation markers (EOMES, brachyury, SOX17, CDX2 and FOXA2) in basigin containing culture, indicating that basigin contributes to the maintenance of the undifferentiated phenotype.

### Example 6

### The HES5:3:3 Employed on Human iPS Cells

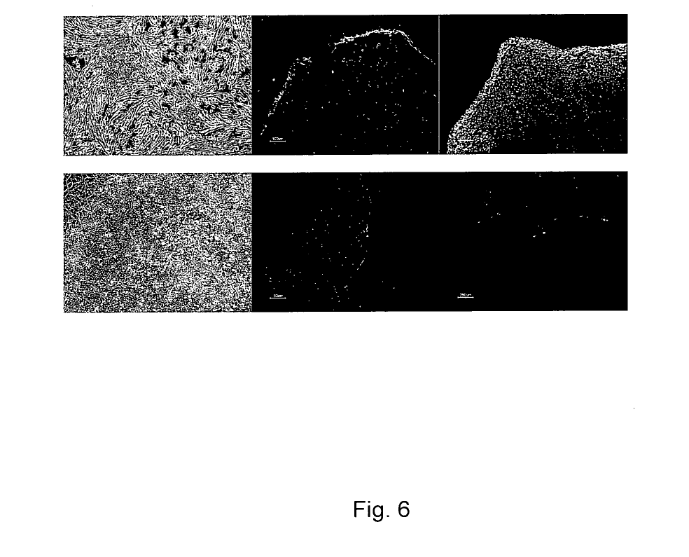

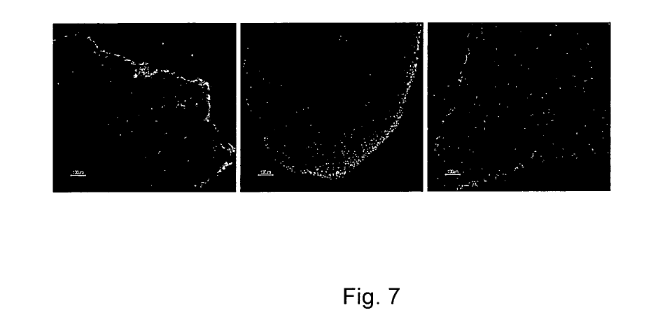

Human iPS cells were thawed onto mEF feeders and initial morphological and immunohistochemical characterization was performed. FIG. 6 shows the morphology of the human iPS cells after thawing, (Left top and below): Thereafter the undifferentiated hiPS cells after thaw on mEFs were immunofluoroscently stained using HES5:3:3 TM (top, mid) Oct-4 (top, right), SSEA-1 (below, mid) and TRA1-60 (below, right). In another experiment the hiPS that were thawed on human Umbilical Wharton Inner Layer (hUWIL) cells and cultured further by employing mechanical passage. After expansion the cells where stained with pluripotent markers (see FIG. 7), such as the HES5:3:3 (top, left) Oct-4 (top, mid), TRA1-60 (top, right). Positive staining with HES5:3:3 were confirmed. These results show that the HES5:3:3 antibody can be used as a tool for human stem cell applications involving stem cells of the group comprising pluripotent stem cells, multipotent stem cells, oligopotent stem cells and/or unipotent cells.

### Example 7

### Analysis of Expression of the Basigin Isoform 1 in Cancer and Normal Tissues with Immunohistochemistry (IHC)

The possibility to detect expression of the target antigen, Basigin isoform 1, in cancer tissues with IHC using MAb HES5:3:3 was analyzed in cancer tissues on Tissue Micro Arrays (TMAs). TMAs were purchased from Super Bio Chips (Seoul, South Korea) Tissue slides were deparaffinated and hydrated by incubation in a dry oven at 60° C. for 1 hour, incubated in xylene 5 times for 4 minutes, incubated in 100%, 95% and 75% ethanol 2 times for 3 minutes and finally immersed in tap water for 5 minutes. Naturally existing peroxidase activity in tissue sections were quenched by incubating slides in 3% H₂O₂ for 5 minutes followed by a 5 minute rinse in tap water. For antigen retrieval, sections were boiled in citrate buffer pH 6.0 {0.42% (w/v) citric acid in H₂O} in a microwave oven for 10 minutes. Slides in citrate buffer were allowed to cool to RT for 45 minutes and were thereafter washed for 5 minutes in H₂O. MAb HES5:3:3 was diluted in Dako Antibody Diluent with Background Reducing Components (Dako A/S, Glostrup, Denmark) to 1 μg/ml, applied to sections and incubated in a humidified chamber for 1.5 hours at room temperature. Excessive antibody and non-specific binding was removed by washing 2 times in TBS, 0.05% Tween 20 for 5 minutes. EnVision+®System-HRP (DAB) (Dako) were used to detect bound antibody according to the manufacturer's protocol and finally cells were counterstained with Mayer's hematoxyline (Like's Modification) (Dako A/S) for 1 second and rinsed in water for 5 minutes.

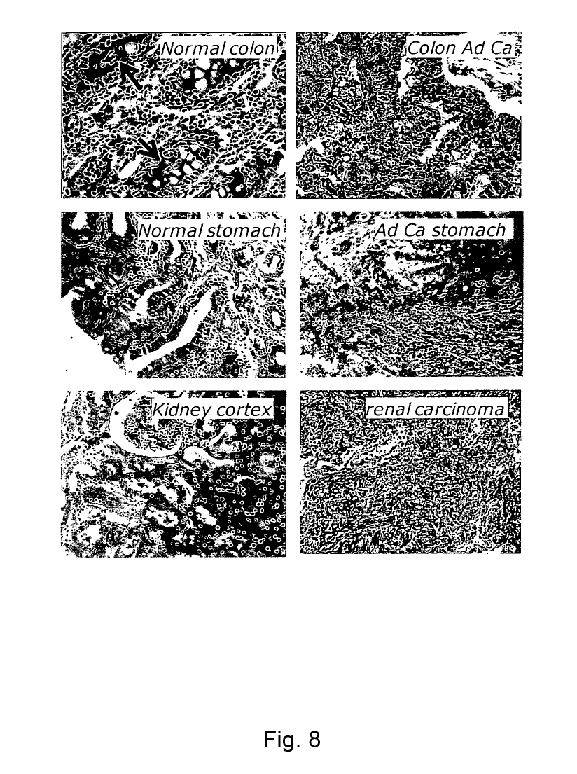

MAb HES5:3:3 was tested on TMAs with cancer tissue sections from different tumours and organs. As can be seen In FIG. 8 HES5:3:3 demonstrated extensive staining towards many carcinomas and Table 2 lists carcinomas of different organs that showed a high degree of positivity. For instance were 11 of 11 ovary serous adenocarcinomas sections positive for HES5:3:3 staining as were tumour cells in 9 of 13 renal cell carcinomas. Most normal tissues were negative for staining with HES5:3:3. In normal colon positive staining was seen to cells deep in the crypts. These cells are “stem cell like” which correlates well with the positivity.

To summarize HES5:3:3 can be used to identify cancer tissue from the group of tissues comprising stomach, colon/rectum, larynx, lung, ovary, kidney, bladder and/or thyroid.

TABLE 2
Results from IHC analysis of cancer tissues with HESS.

| Organ | Cancer | Positive/Total |
|---|---|---|
| Stomach | Adenocarcinoma | 18/30 |
| | GIST | 1/1 |
| Colon/rectum | Adenocarcinoma | 22/27 |
| Larynx | Squamous cell carcinoma | 4/15 |
| Lung | Adenocarcinoma | 3/7 |
| Ovary | Serous carcinoma | 11/11 |
| | Other | 7/11 |
| Kidney | Carcinoma | 9/13 |
| Bladder | Carcinoma | 7/15 |
| Thyroid | Papillary | 8/12 |
| | Follicular | 2/2 |
| | Medullary | 1/1 |

### Comparison of the Specificity of MAb HES5:3:3 and an Anti-Basigin Isoform 2 Antibody to Normal Tissue

The expression of MAb HES5:3:3 in normal tissues was compared to Basigin isoform 2 by staining with an anti-Basigin isoform 2 antibody (cat no sc-80545, Santa Cruz Biotechnology). TMAs with tissue sections from normal organs (Super Bio Chips) were exposed to both antibodies. The antibodies gave staining pattern clearly divergent from each other. Several organs were stained by the Basigin isoform 2 MAb but not MAb HES5:3:3 and IHC data is summarized in Table 3. Even when a particular organ was stained with both antibodies, the staining was against different structures in the tissue section. In colon, for instance, MAb HES5:3:3 stained cells deep in the crypts whereas the Basigin isoform 2 antibody stained cells in the lumen.

The results clearly demonstrate that the expression of the Basigin variant (Basigin isoform 1) targeted MAb HES5:3:3 of the present invention is distinct to that of normal Basigin (Basigin 2, Seq 12 in FIG. 3)

TABLE 3
Summary of IHC results with HES5:3:3 MAb and the Basigin
isoform 2 antibody sc-80545.

| | | Basigin isoform 2 |
|---|---|---|
| Organ | MAb HES5:3:3 | antibody sc-80545 |
| Stomach | negative | positive |
| Liver | negative | positive |
| Stomach | negative | positive |
| Placenta | negative | positive |
| Kidney cortex | negative | positive |
| Cerebellum | negative | positive |

### Example 8

### Determination of MAb HES5:3:3 Epitope on the Unique Part of the Long Splice Variant of Basigin Isoform 1

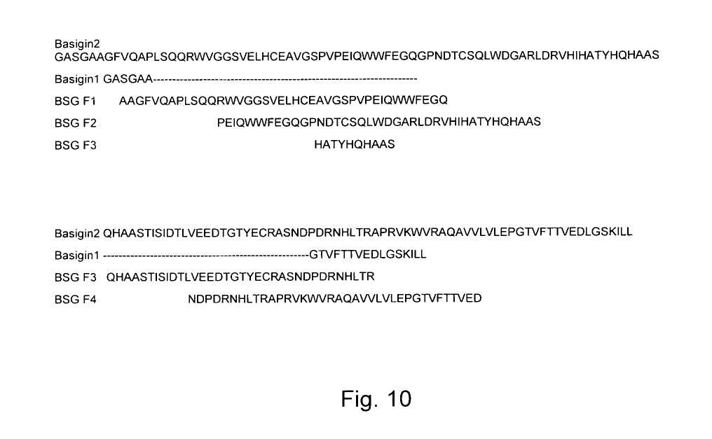

IHC data clearly demonstrated that MAb HES5:3:3 targets a variant of Basigin with a tissue expression pattern distinct from Basigin isoform 2. This indicates that HES5:3:3 targets a splice variant of Basigin. One splice variant of Basigin has an insertion of 115 amino acids (Basigin isoform 1) missing in Basigin isoform 2 (FIGS. 3 and 10). The knowledge of the expression of Basigin isoform 1 is not well explored compared to Basigin isoform 2 that has been shown to be expressed in both normal organs and carcinomas of different origins (see Human protein atlas (http://www.proteinatlas.org/index.php). Since MAb HES5:3:3 clearly does not target the shorter Basigin isoform 2, as judged by difference in tissue expression, it was suspected that the epitope of HES5:3:3 MAb was located within the extra sequence of amino acids of Basigin isoform 1. Therefore phage clones expressing overlapping fragments covering the extra 115 amino acids unique for Basigin isoform 1 were constructed and the reactivity of MAb HES5:3:3 to the phage clones were explored. By this investigation the antibody epitope could be determined to be within the extra 115 amino acids of Basigin isoform 1.

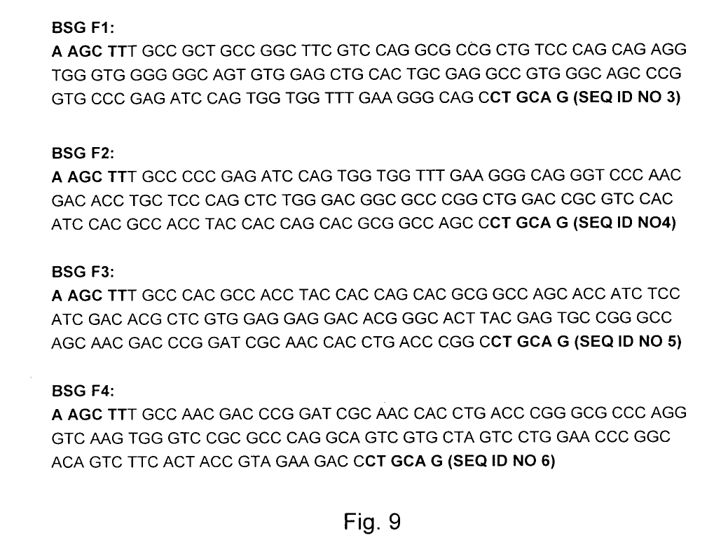

Overlapping DNA fragments (SEQ ID NOS 3-6 in FIGS. 9 and 10) covering the unique parts of Basigin isoform 1 were ordered from GeneScript and were inserted into the phage display vector f88-4. The phage display vector f88-4 (kindly provided by Prof. G. P. Smith, University of Missouri, Columbia, US) and the gene fragments were digested with restriction enzymes HindIII and PstI (GE Healthcare) and were ligated together using T4 DNA ligase (USB, Cleveland, Ohio, US). The DNA constructs were transformed into CaCl₂ competent E. coli JM109 (Stratagene, Cedar Creek, Tex., US). Colony screening was performed by PCR using primers Fwd f88-4: GACCATGGAAAAGTCTTTAGTTCTTAAAG (SEQ ID NO 7) and Rev f88-4: CATAGATCTTTAAGACGCTTTAGAAGTG (SEQ ID NO 8). Phage clones were sequenced and individual clones were grown overnight in LB medium (Sambrook and Russell, 2001) containing 20 μg/ml tetracycline and 1 mM IPTG. Phage particles secreted into the medium were prepared by double polyethylene glycol precipitation. Reactivity of HES5:3:3 MAb to the phage constructs comprising fragments BSG F1-F4 (BSG F1: AAGFVQAPLSQQRWVGGSVELHCEAVGSPVPEIQWWFEGQ (SEQ ID NO 9), BSG F2: PEIQWWFEGQGPNDTCSQLWDGARLDRVHIHATYHQHAAS (SEQ ID NO 10), BSG F3: HATYHQHAASQHAASTISIDTLVEEDTGTYECRASNDPDRN HLTR (SEQ ID NO 11), and BSG F4: NDPDRNHLTRAPRVKWVRAQAVVLVLEPGTVF TTVED (SEQ ID NO 12)) was tested with phage ELISA. HES5:3:3 MAb was coated into Maxisorp wells (Nunc). Phages were diluted in Casein Blocker in PBS (Pierce), added to wells coated with HES5:3:3 MAb and incubated for 30 min at RT. After a washing step, bound phage particles were detected using an HRP labeled anti-M13 antibody (GE Healthcare) and TMB substrate. Signals were measured at 620 nm.

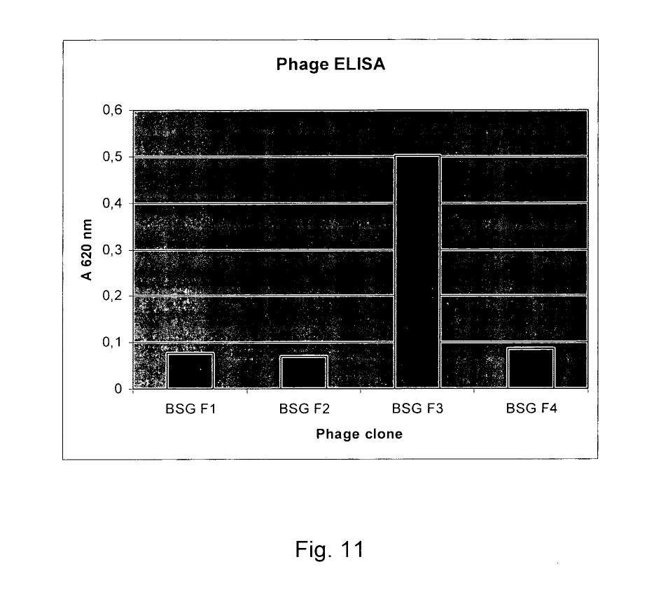

In FIG. 11 it is seen that the phage ELISA demonstrated specific reactivity of MAb HES5:3:3 to fragment BSG F3 having the amino acid sequence HATYHQHAASQHAASTISIDTLVEEDTGTYE CRAS NDPDRNHLTR (SEQ ID NO 11 of FIG. 10). The data demonstrate that MAb HES5:3:3 targets an epitope within the extra 115 amino acids of Basigin isoform 1, and thus consequently that HES5:3:3 MAb is specific for Basigin isoform 1, the long splice variant of Basigin.

SEQUENCE LISTING
Basigin isoform 1:
(SEQ ID NO: 1)
MAAALFVLLG FALLGTHGAS GAAGFVQAPL SQQRWVGGSV ELHCEAVGSP
VPEIQWWFEG QGPNDTCSQL WDGARLDRVH IHATYHQHAA STISIDTLVE
EDTGTYECRA SNDPDRNHLT RAPRVKWVRA QAVVLVLEPG TVFTTVEDLG
SKILLTCSLN DSATEVTGHR WLKGGVVLKE DALPGQKTEF KVDSDDQWGE
YSCVFLPEPM GTANIQLHGP PRVKAVKSSE HINEGETAML VCKSESVPPV
TDWAWYKITD SEDKALMNGS ESRFFVSSSQ GRSELHIENL NMEADPGQYR
CNGTSSKGSD QAIITLRVRS HLAALWPFLG IVAEVLVLVT IIFIYEKRRK PEDVLDDDDA
GSAPLKSSGQ HQNDKGKNVR QRNSS
Basigin isoform 2:
(SEQ ID NO: 2)
MAAALFVLLG FALLGTHGAS GAAG TVFTTVEDLG SKILLTCSLN DSATEVTGHR
WLKGGVVLKE DALPGQKTEF KVDSDDQWGE YSCVFLPEPM GTANIQLHGP
PRVKAVKSSE HINEGETAML VCKSESVPPV TDWAWYKITD SEDKALMNGS
ESRFFVSSSQ GRSELHIENL NMEADPGQYR CNGTSSKGSD QAIITLRVRS
HLAALWPFLG IVAEVLVLVT IIFIYEKRRK PEDVLDDDDA GSAPLKSSGQ
HQNDKGKNVR QRNSS
BSG F1 (DNA sequence phage insert)
(SEQ ID NO 3)
A AGC TTT GCC GCT GCC GGC TTC GTC CAG GCG CCG CTG TCC CAG CAG AGG
TGG GTG GGG GGC AGT GTG GAG CTG CAC TGC GAG GCC GTG GGC AGC CCG
GTG CCC GAG ATC CAG TGG TGG TTT GAA GGG CAG CCT GCA G
BSG F2 (DNA sequence phage insert):
(SEQ ID NO 4)
A AGC TTT GCC CCC GAG ATC CAG TGG TGG TTT GAA GGG CAG GGT CCC AAC
GAC ACC TGC TCC CAG CTC TGG GAC GGC GCC CGG CTG GAC CGC GTC CAC
ATC CAC GCC ACC TAC CAC CAG CAC GCG GCC AGC CCT GCA G
BSG F3 (DNA sequence phage insert):
(SEQ ID NO 5)
A AGC TTT GCC CAC GCC ACC TAC CAC CAG CAC GCG GCC AGC ACC ATC TCC
ATC GAC ACG CTC GTG GAG GAG GAC ACG GGC ACT TAO GAG TGC CGG GCC
AGC AAC GAC CCG GAT CGC AAC CAC CTG ACC CGG CCT GCA G
BSG F4 (DNA sequence phage insert):
(SEQ ID NO 6)
A AGC TTT GCC AAC GAC CCG GAT CGC AAC CAC CTG ACC CGG GCG CCC AGG
GTC AAG TGG GTC CGC GCC CAG GCA GTC GTG CTA GTC CTG GAA CCC GGC
ACA GTC TTC ACT ACC GTA GAA GAC CCT GCA G
Fwd f88-4:
(SEQ ID NO 7)
GACCATGGAAAAGTCTTTAGTTCTTAAAG
Rev f88-4:
(SEQ ID NO 8)
CATAGATCTTTAAGACGCTTTAGAAGTG
BSG F1 (fragment)
(SEQ ID NO 9)
AAGFVQAPLSQQRWVGGSVELHCEAVGSPVPEIQWWFEGQ
BSG F2 (fragment)
(SEQ ID NO 10)
PEIQWWFEGQGPNDTCSQLWDGARLDRVHIHATYHQHAAS
BSG F3 (fragment)
(SEQ ID NO 11)
HATYHQHAASQHAASTISIDTLVEEDTGTYE CRAS NDPDRNHLTR
BSG F4 (fragment)
(SEQ ID NO 12)
NDPDRNHLTRAPRVKWVRAQAVVLVLEPGTVFTTVED

1-51. (canceled)

52. Use of a polypeptide characterized in that it is represented by the following amino acid sequence:
MAAALFVLLG FALLGTHGAS GAAGFVQAPL SQQRWVGGSV ELHCEAVGSP VPEIQWWFEG QGPNDTCSQL WDGARLDRVH IHATYHQHAA STISIDTLVE EDTGTYECRA SNDPDRNHLT RAPRVKWVRA QAVVLVLEPG TVFTTVEDLG SKILLTCSLN DSATEVTGHR WLKGGVVLKE DALPGQKTEF KVDSDDQWGE YSCVFLPEPM GTANIQLHGP PRVKAVKSSE HINEGETAML VCKSESVPPV TDWAWYKITD SEDKALMNGS ESRFFVSSSQ GRSELHIENL NMEADPGQYR CNGTSSKGSD QAIITLRVRS HLAALWPFLG IVAEVLVLVT IIFIYEKRRK PEDVLDDDDA GSAPLKSSGQ HQNDKGKNVR QRNSS (SEQ ID NO 1), or a polypeptide consisting of a partial sequence of an amino acid sequence represented by SEQ ID NO 1
as a tool for human stem cell applications involving human pluripotent stem cells.

53. Use according to claim 52, wherein the partial sequence is one or more of the following sequences AAGFVQAPLSQQRWVGGSVELHCEAVGSPVPEIQWWFEGQ (SEQ ID NO 9), PEIQWWFEGQGPNDTCSQLWDGARLDRVHIHATYHQHAAS (SEQ ID NO 10), or NDPDRNHLTRAPRVKWVRAQAVVLVLEPGTVFTTVED (SEQ ID NO 12).

54. Use according to claim 52 as a marker to selectively mark, sort or separate early differentiated cell types derived from human pluripotent stem cells (hPSC).

55. Use according to claim 52 as a marker to selectively mark, sort or separate human pluripotent stem cells (hPSC).

56. Use according to claim 52 as a marker to selectively mark, sort or separate undifferentiated human pluripotent stem cells (hPSC).

57. Use according to claim 52 as a media supplement or culture vessel coating for the enhanced survival and adhesion of human pluripotent stem cells (hPSC) during culturing.

58. Use according to claim 52 for the down regulation of the differentiation process in human pluripotent stem cells (hPSC) in non-supportive culture conditions.


SEQUENCE LISTING (12 sequences)

SEQ ID NO: 1 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 2 - protein, 269 aa, Homo sapiens
MAAALFVLLGFALLGTHGASGAAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 3 - DNA, 137 bp, Homo sapiens
aagctttgccgctgccggcttcgtccaggcgccgctgtcccagcagaggtgggtggggggcagtgtggagctgcactgcgaggccgtgggcagcccggtgcccgagatccagtggtggtttgaagggcagcctgcag

SEQ ID NO: 4 - DNA, 137 bp, Homo sapiens
aagctttgcccccgagatccagtggtggtttgaagggcagggtcccaacgacacctgctcccagctctgggacggcgcccggctggaccgcgtccacatccacgccacctaccaccagcacgcggccagccctgcag

SEQ ID NO: 5 - DNA, 137 bp, Homo sapiens
aagctttgcccacgccacctaccaccagcacgcggccagcaccatctccatcgacacgctcgtggaggaggacacgggcacttacgagtgccgggccagcaacgacccggatcgcaaccacctgacccggcctgcag

SEQ ID NO: 6 - DNA, 128 bp, Homo sapiens
aagctttgccaacgacccggatcgcaaccacctgacccgggcgcccagggtcaagtgggtccgcgcccaggcagtcgtgctagtcctggaacccggcacagtcttcactaccgtagaagaccctgcag

SEQ ID NO: 7 - DNA, 29 bp, Artificial Sequence
  Primer
gaccatggaaaagtctttagttcttaaag

SEQ ID NO: 8 - DNA, 28 bp, Artificial Sequence
  Primer
catagatctttaagacgctttagaagtg

SEQ ID NO: 9 - protein, 40 aa, Homo sapiens
AAGFVQAPLSQQRWVGGSVELHCEAVGSPVPEIQWWFEGQ

SEQ ID NO: 10 - protein, 40 aa, Homo sapiens
PEIQWWFEGQGPNDTCSQLWDGARLDRVHIHATYHQHAAS

SEQ ID NO: 11 - protein, 45 aa, Homo sapiens
HATYHQHAASQHAASTISIDTLVEEDTGTYECRASNDPDRNHLTR

SEQ ID NO: 12 - protein, 37 aa, Homo sapiens
NDPDRNHLTRAPRVKWVRAQAVVLVLEPGTVFTTVED